(19) United States
(12) Patent Application Publication
(10) Pub. No.: US 2011/0112446 A1
(43) Pub. Date: May 12, 2011

(54) ULTRASONIC TREATMENT APPARATUS AND ULTRASONIC TREATMENT METHOD

(75) Inventor: Ken HARA, Tokyo (JP)

(73) Assignee: OLYMPUS MEDICAL SYSTEMS CORP., Tokyo (JP)

(21) Appl. No.: 13/009,421

(22) Filed: Jan. 19, 2011

Related U.S. Application Data
(63) Continuation of application No. 11/171,685, filed on Jun. 30, 2005.

(51) Int. Cl. A61N 7/00

(52) U.S. Cl. 601/2

(57) ABSTRACT

Provided is an ultrasonic treatment apparatus which includes a drive signal generating unit (such as a FPGA circuit), a detecting unit (output detecting circuit), a range setting unit (condition setting circuit), and a frequency controller (frequency changing circuit). The drive signal generating unit generates a drive signal of a predetermined oscillating frequency by performing a frequency sweep. The detecting unit detects information on a state of the drive signal. The range setting unit sets a range of frequency sweep based on identification information of an ultrasonic transducer. The frequency controller performs a frequency control in the range of frequency sweep based on the information on the state of the drive signal.

### CROSS-REFERENCE TO RELATED APPLICATION

The present application is a continuation under 37 C.F.R. §1.53(b) of prior application Ser. No. 11/171,685 filed Jun. 30, 2005, entitled ULTRASONIC TREATMENT APPARATUS AND ULTRASONIC TREATMENT METHOD, the entire contents of which are incorporated by reference herein.

### BACKGROUND OF THE INVENTION

1. Field of the Invention

The present invention relates to an ultrasonic treatment apparatus that serves to drive an ultrasonic transducer and a method of performing an ultrasonic treatment.

2. Description of the Related Art

Conventionally, various ultrasonic treatment apparatuses are proposed which employ an ultrasonic transducer that generates ultrasonic vibrations. One of such known ultrasonic treatment apparatus is inserted into a body cavity of a subject and serves to realize observation of a living tissue in the body cavity and various treatments such as incision or coagulation of the living tissue.

In such an ultrasonic treatment apparatus, it is desirable that an electric energy is efficiently converted into a mechanical energy, i.e., ultrasonic vibrations of the ultrasonic transducer.

### SUMMARY OF THE INVENTION

An ultrasonic treatment apparatus according to the present invention includes a drive signal generating unit that generates a drive signal for driving an ultrasonic transducer with a predetermined oscillating frequency by a frequency sweep; a detecting unit that detects state information of the drive signal to the ultrasonic transducer; a range setting unit that sets a sweeping range of frequencies at the frequency sweep based on identification information of the ultrasonic transducer; and a frequency controller that performs a frequency control of the drive signal generating unit so that the drive signal generating unit performs the frequency sweep of the drive signal by switching the frequencies in the sweeping range based on the state information.

An ultrasonic treatment apparatus according to the present invention includes a drive signal generating unit that generates a drive signal for driving an ultrasonic transducer with a predetermined oscillating frequency by a frequency sweep, the ultrasonic transducer generating ultrasonic vibrations; a detecting unit that detects state information of the drive signal; an interval setting unit that sets a sweeping interval of frequencies at the frequency sweep based on identification information of the ultrasonic transducer; and a frequency controller that performs a frequency control of the drive signal generating unit so that the drive signal generating unit performs the frequency sweep by switching the frequencies at the sweeping interval based on the state information.

An ultrasonic treatment apparatus according to the present invention includes a drive signal generating unit that generates a drive signal for driving an ultrasonic transducer with a predetermined oscillating frequency by a frequency sweep, the ultrasonic transducer generating ultrasonic vibrations; a detecting unit that detects state information of the drive signal; a switching time setting unit that sets a switching time of frequencies at the frequency sweep based on identification information of the ultrasonic transducer; and a frequency controller that performs a frequency control of the drive signal generating unit so that the drive signal generating unit performs the frequency sweep by switching the frequency at the switching time-based on the state information.

The ultrasonic treatment apparatus according to the present invention may further include an interval setting unit that sets a sweeping interval of frequencies at the frequency sweep based on the identification information. The frequency controller performs the frequency control of the drive signal generating unit so that the drive signal generating unit performs the frequency sweep-by switching the frequencies at the sweeping interval in the sweeping range based on the state information.

The ultrasonic treatment apparatus according to the present invention may further include a switching time setting unit that sets a switching time of the frequencies at the frequency sweep based on the identification information. The frequency controller performs the frequency control of the drive signal generating unit so that the drive signal generating unit performs the frequency sweep by switching the frequency at the switching time in the sweeping range based on the state information.

The ultrasonic treatment apparatus according to the present invention may further include an interval setting unit that sets a sweeping interval of frequencies at the frequency sweep based on the identification information; and a switching time setting unit that sets a switching time of frequencies at the frequency sweep based on the identification information. The frequency controller performs the frequency control of the drive signal generating unit so that the drive signal generating unit performs the frequency sweep by switching the frequencies at the switching time at the sweeping interval in the sweeping range based on the state information.

The ultrasonic treatment apparatus according to the present invention may further include a switching time setting unit that sets a switching time of the frequencies at the frequency sweep-based on the identification information. The frequency controller performs the frequency control of the drive signal generating unit so that the drive signal generating unit performs the frequency sweep by switching the frequencies at the switching time at the sweeping interval based on the state information.

In the ultrasonic treatment apparatus according to the present invention, the detecting unit may phase information of the drive signal as the state information.

A method of ultrasonic treatment, according to the present invention, includes generating a drive signal for driving an ultrasonic transducer with a predetermined oscillating frequency by a frequency sweep of the drive signal to enable a generation of ultrasonic vibrations from the ultrasonic transducer; detecting state information of the drive signal; setting a sweeping range of frequencies at the frequency sweep based on identification information of the ultrasonic transducer; and performing a frequency control of the drive signal so that the frequency sweep is performed by switching the frequencies in the sweeping range based on the state information.

A method of ultrasonic treatment according to the present invention includes generating a drive signal for driving an ultrasonic transducer with a predetermined oscillating frequency by a frequency sweep of the drive signal to enable a generation of ultrasonic vibrations from the ultrasonic transducer; detecting state information of the drive signal; setting a sweeping interval of frequencies at the frequency sweep based on identification information of the ultrasonic transducer; and performing a frequency control of the drive signal so that the frequency sweep is performed by switching the frequencies at the sweeping interval based on the state information.

A method of ultrasonic treatment according to the present invention includes generating a drive signal for driving an ultrasonic transducer with a predetermined oscillating frequency by a frequency sweep of the drive signal to enable a generation of ultrasonic vibrations from the ultrasonic transducer; detecting state information of the drive signal; setting a switching time of frequencies at the frequency sweep based on identification information of the ultrasonic transducer; and performing a frequency control of the drive signal so that the frequency sweep is performed by switching the frequencies at the switching time based on the state information.

The method of ultrasonic treatment according to the present invention may further include setting a sweeping interval of the frequencies at the frequency sweep based on the identification information. The performing the frequency control may include performing the frequency control so that the frequency sweep is performed by switching the frequencies at the sweeping interval in the sweeping range based on the state information.

The method of ultrasonic treatment according to the present invention may further include setting a switching time of the frequencies at the frequency sweep based on the identification information. The performing the frequency control may include performing the frequency control so that the frequency sweep is performed by switching the frequencies at the switching time in the sweeping range based on the state information.

The method of ultrasonic treatment according to the present invention may further include setting a sweeping interval of the frequencies at the frequency sweep based on the identification information; and

setting a switching time of the frequencies at the frequency sweep based on the identification information. The performing the frequency control may include performing the frequency control so that the frequency sweep is performed by switching the frequencies at the switching time at the sweeping interval in the sweeping range based on the state information.

The method of ultrasonic treatment according to the present invention may further include setting a switching time of the frequencies at the frequency sweep based on the identification information. The performing the frequency control may include performing the frequency control so that the frequency sweep is performed by switching the frequencies at the switching time at the sweeping interval based on the state information.

In the method of ultrasonic treatment according to the present invention the detecting may include detecting phase information of the drive signal as the state information.

The above and other objects, features and advantages of the present invention will be further clarified in a following detailed description of the present invention in connection with the accompanying drawings.

### BRIEF DESCRIPTION OF THE DRAWINGS

FIG. 1 is a block diagram of an overall structure of an ultrasonic treatment apparatus;

FIG. 2 is a block diagram of a structure of a FPGA circuit shown in FIG. 1;

FIG. 3 is a diagram illustrating a detection of a resonance frequency;

FIG. 4 is a flowchart illustrating an operation of resonance frequency detection by the ultrasonic treatment apparatus shown in FIG. 1;

FIG. 5 is a block diagram of a structure of a SIN waveform generating circuit shown in FIG. 2;

FIG. 6 is a flowchart illustrating operations of a SIN wave address generating unit and an address quartering unit shown in FIG. 5; and

FIG. 7 is a flowchart illustrating an operation of a code switching unit shown in FIG. 5.

### DETAILED DESCRIPTION

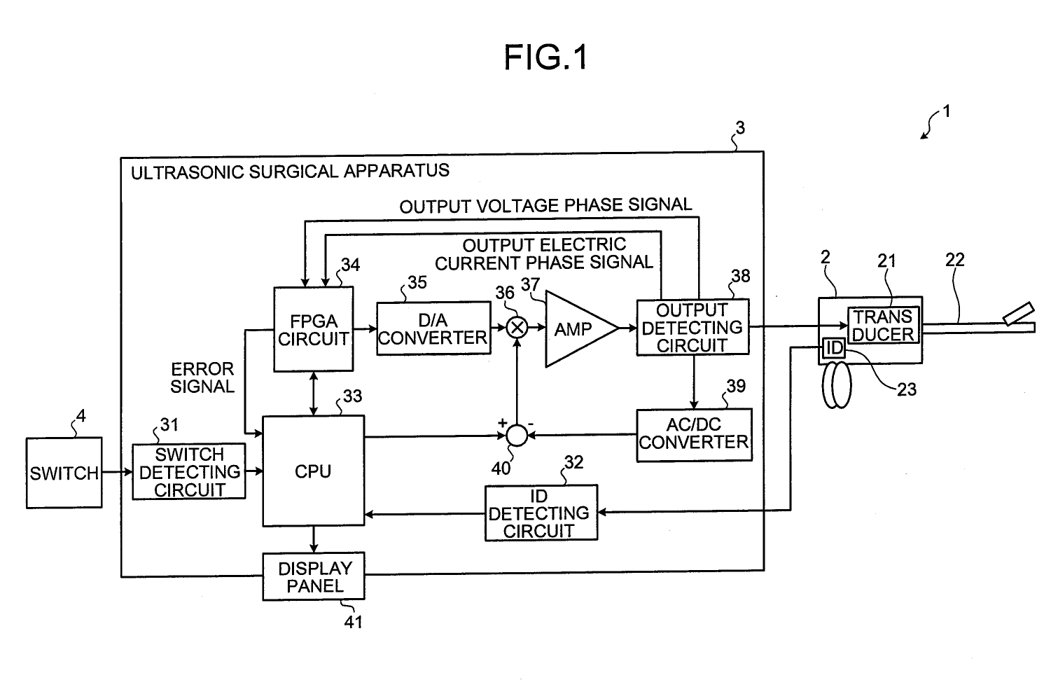

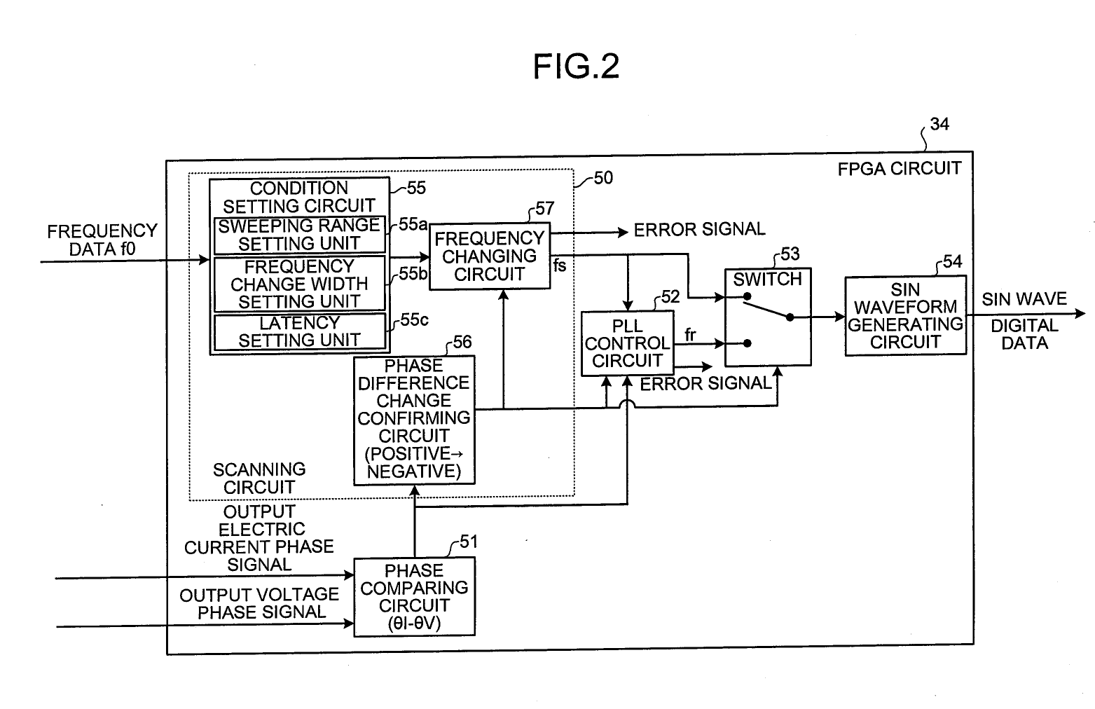

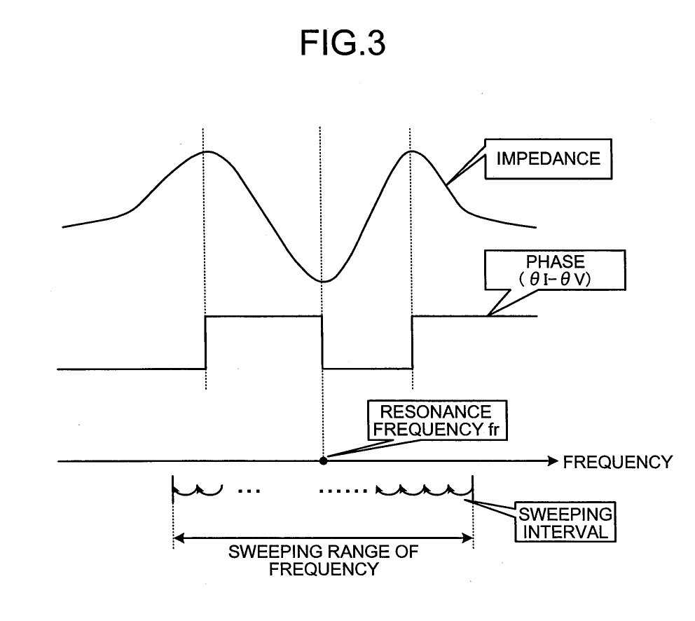

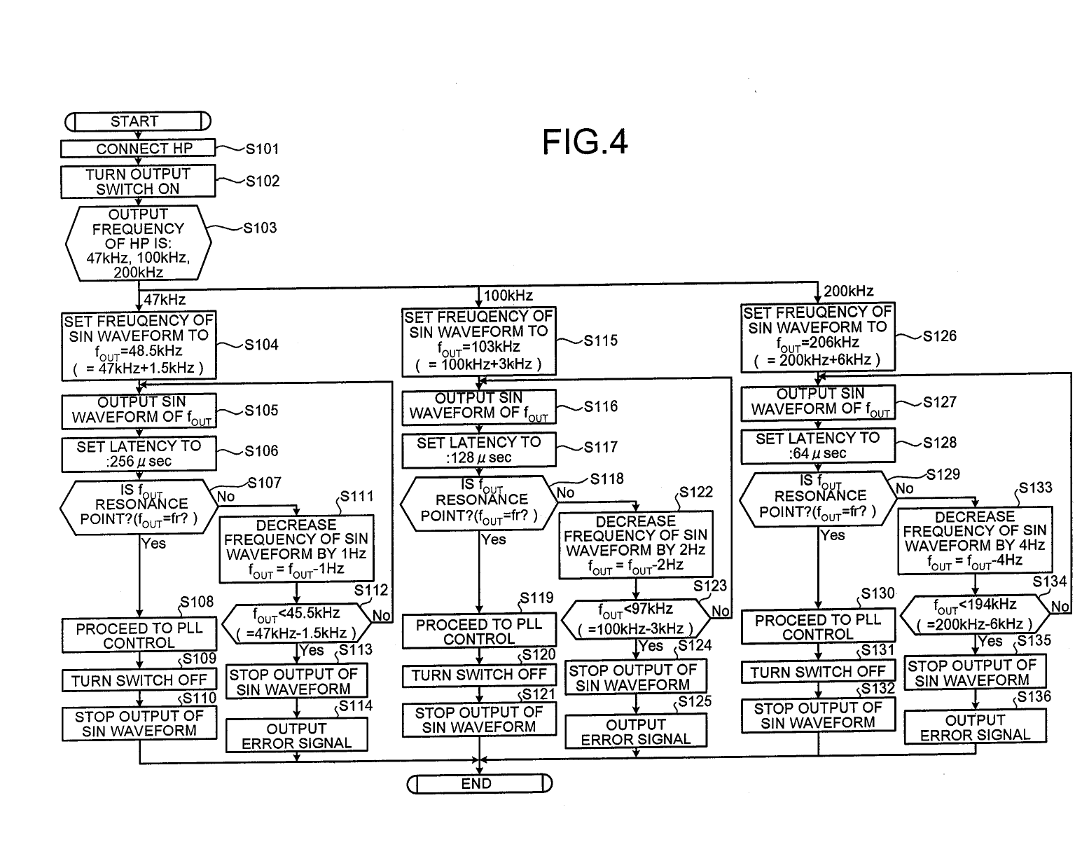

FIGS. 1 to 4 are drawings related to an exemplary embodiment of an ultrasonic treatment apparatus according to the present invention. Among these drawings, FIG. 1 is a block diagram of an overall structure of the ultrasonic treatment apparatus. FIG. 2 is a block diagram of a structure of a FPGA circuit shown in FIG. 1. FIG. 3 is a diagram illustrating a detection of a resonance frequency. FIG. 4 is a flowchart illustrating an operation of resonance frequency detection by the ultrasonic treatment apparatus shown in FIG. 1.

U.S. Pat. No. 6,019,775 discloses an apparatus which generates a frequency signal for driving an ultrasonic transducer based on data which is stored in a read only memory (ROM) and associated with a mechanical resonance frequency of a handpiece, amplifies the frequency signal at a power amplifier, to output the result to the ultrasonic transducer. Further, United States Patent Application Laid-Open No. 2004-0102709 discloses an initial resonance frequency detector which increases a sweeping speed for initial resonance frequency detection when a phase difference amount between ultrasonic outputs is large and an oscillating frequency of a drive frequency setting signal is far from the initial resonance frequency, and decreases the sweeping speed for the initial resonance frequency detection when the phase difference amount between the ultrasonic outputs is small and the oscillating frequency of the drive frequency setting signal is close to the initial resonance frequency.

In recent years, inherent frequencies of the ultrasonic transducers vary according to the use of the ultrasonic transducers and are distributed in a wide range of frequency band, and accordingly, a provision of an apparatus that can detect each of the plural inherent frequencies in the wide range of frequency band is required. Such an apparatus is required to change a detection condition, such as a frequency sweeping range, as appropriate according to a frequency band of a detection target, rather than to set a fixed condition. For example, at a detection of a low frequency, the frequency sweeping range needs to be set narrower than that at a detection of a high frequency, in order to prevent an erroneous detection of a resonance point. The conventional techniques disclosed in the cited patent documents, however, set the fixed frequency sweeping range regardless of the level of resonance frequency, and hence, when plural resonance frequencies in a wide frequency band need to be detected, are unable to deal with the detection of all resonance frequencies sufficiently.

The present invention solves at least the problems in the above mentioned patent documents.

An ultrasonic treatment apparatus 1, as shown in FIG. 1, includes a handpiece 2 that performs an ultrasonic treatment, an ultrasonic surgical apparatus 3 that supplies an ultrasonic output to the handpiece 2, and an output switch 4 that controls the ultrasonic output of the ultrasonic surgical apparatus 3.

The handpiece 2 includes an ultrasonic transducer 21 having an inherent frequency band, a probe 22 with various shapes, and an ID storing unit 23 that stores identification information specific to the ultrasonic transducer 21. The handpiece 2 is detachably attached to the ultrasonic surgical apparatus 3 via a connector not shown, for example. The ultrasonic transducer 21 has a different oscillating frequency according to a type thereof, and may have a frequency band of 47 kilohertz (kHz), 100 kHz, or 200 kHz, for example. The probe 22, due to manufacturing variations attributable to a manufacturing process, has a variation in a range of a few kilohertz from the initial resonance frequency of the manufactured probe 22. In the handpiece 2, an electric signal (electric energy) supplied from the ultrasonic surgical apparatus 3 is converted into a mechanical vibrations (mechanical energy) by the ultrasonic transducer 21, and a treatment in the body cavity of the subject is realized with mechanical vibrations of the probe 22 connected to the ultrasonic transducer 21. The ultrasonic surgical apparatus 3 according to the embodiment is set so that an initial resonance frequency fᵣ can be obtained at a small impedance as shown in FIG. 3 for an efficient conversion of an electric energy into a mechanical energy in the form of ultrasonic vibrations of the ultrasonic transducer.

The ultrasonic surgical apparatus 3 includes a switching detecting circuit 31 that detects an ON/OFF operation of the output switch 4 such as a footswitch, an ID detecting circuit 32 that detects identification information from the ID storing unit 23, a central processing unit (CPU) 33 that receives detection signals from the detecting circuits 31 and 32 as inputs, a Field Programmable Gate Array (FPGA) circuit 34 that is controlled by the CPU, a digital-analog converter 35 that performs digital/analog conversion on an output from the FPGA circuit 34, a multiplier 36 that multiplies an output signal from the D/A converter 35 and an output signal from an adder 40, an amplifier 37 that amplifies an output from the multiplier 36, an output detecting circuit 38 that receives an output from the amplifier 37 as an input, an AC/DC converter 39 that performs an AC/DC conversion on an output signal from the output detecting circuit 38, the adder 40 that adds up an output signal from the CPU and an output signal from the AC/DC converter 39, and a display panel 41 that is connected to the CPU 33 and displays various information. The FPGA circuit 34, the D/A converter 35, the multiplier 36, and the amplifier 37 form a drive signal generating unit according to the present invention. Further, the multiplier 36, the amplifier 37, the output detecting circuit 38, the AC/DC converter 39, and the adder 40 form a feedback circuit for performing a constant current control. Here, the FPGA circuit 34 can be formed from an analog circuit, for example, or alternatively, formed from a DSP or a CPU.

The output detecting circuit 38 has a function to supply the output from the amplifier 37 to the ultrasonic transducer 21, and a function as a detecting unit that detects phases of an output voltage and an output electric current which represent a voltage and a state of voltage output based on the output from the amplifier 37. The output voltage phase signal and the output electric current phase signal as detected are supplied to the FPGA circuit 34 from the output detecting circuit 38. Further, the output detecting circuit 38 outputs information on an output setting value as detected (negative electric current value) to the adder 40.

The adder 40 receives information on the output setting value (electric current value) output from the CPU 33 and information on the output setting value output from the AC/DC converter 39 for an output setting at a time of resonance frequency detection, and outputs a result of summation of the output setting values to the multiplier 36. The multiplier 36 performs a constant current control on the output from the D/A converter 35 based on the result of summation output from the adder 40.

The CPU 33 stores frequency data f₀ on the resonance frequency corresponding to the identification information specific to the ultrasonic transducer, and supplies frequency data f₀ corresponding to the identification information from the ID detecting circuit 32 to the FPGA circuit 34 as an output.

The FPGA circuit 34, as shown in FIG. 2, includes a scanning circuit 50 that performs a frequency sweep based on the frequency data f₀ supplied from the CPU 33, a phase comparing circuit 51 that performs a phase comparison based on the output electric current phase signal and the output voltage phase signal supplied from the output detecting circuit 38, a PLL control circuit 52 that performs a PLL control based on a drive signal with frequency fₛ (hereinafter referred to as a drive signal fₛ) supplied from the scanning circuit 50 and outputs a drive signal with a resonance frequency fᵣ (hereinafter referred to as a drive signal fᵣ), a switch 53 that performs a switching operation according to a confirmation signal from the scanning circuit 50, and a SIN waveform generating circuit 54 that generates a sine waveform based on one of the drive signal fₛ and the drive signal fᵣ. According to the embodiment, the scanning circuit 50 has a function of bringing the oscillating frequency closer to the resonance frequency, whereas the PLL control circuit 52 has a function of locking the oscillating frequency to the resonance frequency.

The scanning circuit 50 includes a condition setting circuit 55 that sets various conditions based on the frequency data f0 supplied as an input, a phase difference change confirming circuit 56 that confirms a change in phase difference based on a result of comparison from the phase comparing circuit 51, and a frequency changing circuit 57 that serves as a frequency controller that performs a frequency sweep control to change the frequency based on various conditions set by the condition setting circuit 55. The condition setting circuit 55 is a circuit for setting a condition for performing the frequency sweep. The condition setting circuit 55 includes a sweeping range setting unit 55a that serves as a range setting unit that sets a sweeping range that is an upper limit and a lower limit of frequency to be swept, a frequency change width setting unit 55b that serves as an interval setting unit that sets a change width (sweeping interval) of the frequency to be swept, and a latency setting unit 55c that serves as a switching time setting unit that sets a latency (switching time) at a time of change of one frequency to another frequency.

The condition setting circuit 55 supplies information of setting conditions dealt by the sweeping range setting unit 55a, the frequency change width setting unit 55b, and the latency setting unit 55c based on the frequency data fₒ, which is supplied from the CPU 33, to the frequency changing circuit 57. The setting units 55a to 55c respectively set the setting conditions by switching the sweeping range for the frequency sweep, the frequency change width, and the latency according to the output frequency band indicated by the frequency data f₀, so that the condition for each output frequency band is uniform. For example, when the output frequency band in the frequency data f₀ is 47 kHz, the sweeping range setting unit 55a outputs setting information on the sweeping range of 47 (kHz)±1.5 (kHz), the frequency change width setting unit 55b outputs setting information on the change width of 1 Hz, and the latency setting unit 55c outputs setting information on the latency of 256 μsec. Further, when the output frequency band is 100 kHz, the sweeping range setting unit 55a outputs setting information on the sweeping range of 100 (kHz)±3.0 (kHz), the frequency change width setting unit 55b outputs setting information on the change width of 2 Hz, and the latency setting unit 55c outputs setting information on the latency of 128 μsec. Still further, when the output frequency band is 200 kHz, the sweeping range setting unit 55a outputs setting information on the sweeping range of 200 (kHz)±6.0 (kHz), the frequency change width setting unit 55b outputs setting information on the change width of 4 Hz, and the latency setting unit 55c outputs setting information on the latency of 64 μsec. Thus, the condition setting circuit 55 sets a smaller sweeping range and frequency change width, and a longer latency for a low output frequency band since the sine waveform of the frequency is indicated as a smooth waveform on a time axis, whereas sets a larger sweeping range and frequency change width, and a shorter latency for a high output frequency band since the sine waveform of the frequency is indicated as a sharp waveform on the time axis.

The frequency changing circuit 57 performs a frequency sweep of the drive signal fₛ based on the setting information of respective sweep conditions supplied from the respective setting units 55a to 55c of the condition setting circuit 55.

The phase comparing circuit 51 compares the phase of the output electric current and the phase of the output voltage based on the supplied output electric current phase signal and the output voltage phase signal, and outputs a signal showing a phase difference (θ_{I}−θ_{V}) shown in FIG. 3 to the PLL control circuit 52 and the phase difference change confirming circuit 56. Here, θ_{I} represents a phase of an output electric current I detected by the output detecting circuit 38 whereas θ_{V} represents a phase of an output voltage V detected by the output detecting circuit 38.

The phase difference change confirming circuit 56 confirms a state of change of the phase from a positive to a negative based on the phase difference of (θ_{I}−θ_{V}) which is a result of comparison supplied from the phase comparing circuit 51. The phase difference change confirming circuit 56, when the phase difference changes from a positive to a negative, outputs a state signal indicating the changed state to the frequency changing circuit 57, the PLL control circuit 52, and the switch 53. When the phase difference change confirming circuit 56 outputs the state signal, the switch 53 operates as to connect the PLL control circuit 52 and the SIN waveform generating circuit 54, and then the frequency sweep operation transits from the frequency changing circuit 57 that receives the state signal to the PLL control circuit 52. Then the PLL control circuit 52 performs the frequency sweep on the drive signal, and locks a current oscillating frequency as the resonance frequency fᵣ when the phase difference (θ_{I}−θ_{V}) shown in FIG. 3 falls from a positive value to “zero,” and outputs the drive signal fᵣ corresponding to the resonance frequency to the SIN waveform generating circuit 54.

Next, the operation of the resonance frequency detection by the ultrasonic treatment apparatus 1 will be described with reference to the flowchart of FIG. 4. In the description of the embodiment, the output frequency of the handpiece 2 (oscillating frequency of the ultrasonic transducer 21) is assumed to be either 47 kHz, 100 kHz, or 200 kHz.

In FIG. 4, first the handpiece 2 is connected to the ultrasonic surgical apparatus 3 (step S101), to turn the output switch 4 ON (step S102). Then, the CPU 33 retrieves identification information specific to the ultrasonic transducer 21 to determine whether the output frequency of the handpiece 2 is 47 kHz, 100 kHz, or 200 kHz, and outputs the frequency data f₀ indicating the pertinent output frequency to the FPGA circuit 34 (step S103).

In step S103, if the output frequency of the handpiece 2 is 47 kHz according to the identification information, the CPU 33 outputs the frequency data f₀ indicating 47 kHz to the FPGA circuit 34, and then the scanning circuit 50 of the FPGA circuit 34 sets frequency fₒᵤₜ of the SIN waveform (drive signal) to an upper limit of the sweeping range, i.e., 48.5 (kHz)=47 (kHz)+1.5 (kHz) based on the frequency data f₀ (step S104), and the SIN waveform generating circuit 54 outputs a SIN waveform of the above-mentioned frequency (step S105).

Then, the scanning circuit 50 sets the latency to 256 μsec (step S106), while maintaining the output of the SIN waveform, and determines whether the frequency fₒᵤₜ is the resonance frequency fₛ of the resonant point (step S107).

When the frequency fₒᵤₜ matches with the resonance frequency fₛ in step S107 (Yes in step S107), the operation of resonance frequency detection transits from the scanning circuit 50 to the PLL control circuit 52, and the detection of resonance frequency, i.e., the operation of frequency sweep is performed (step S108), thereafter the output switch 4 is turned OFF (step S109) and the output of the SIN waveform is stopped (step S110) to end the operation. On the other hand, when the frequency fₒᵤₜ does not match with the resonance frequency fₛ in step S107 (No in step S107), the scanning circuit 50 decreases the frequency fₒᵤₜ of the SIN waveform by 1 Hz to render fₒᵤₜ=fₒᵤₜ−1 (Hz) (step S111), and determines whether the frequency fₒᵤₜ of the SIN waveform is lower than the lower limit of the sweeping range, i.e., 45.5 (kHz)=47 (kHz)−1.5 (kHz) or not (step S112).

When the frequency fₒᵤₜ of the SIN waveform does not reach 45.5 kHz in step S112 (No in step S112), the process returns to step S105 to output the SIN waveform of a corresponding frequency fₒᵤₜ=48.5 (kHz)−1 (Hz), and the operation described above is repeated. On the other hand, when the frequency fₒᵤₜ of the SIN waveform is lower than 45.5 kHz, the FPGA circuit 34 stops the output of the SIN waveform (step S113), outputs an error signal to display an error message, for example, on the display panel 41 (step S114).

When the output frequency of the handpiece 2 is 100 kHz according to the identification information in step S103, similarly to the above description, the CPU 33 outputs the frequency data f₀ indicating 100 kHz to the FPGA circuit 34, and the scanning circuit 50 of the FPGA circuit 34 sets the frequency fₒᵤₜ of the SIN waveform (drive signal) to the upper limit of the sweeping range, i.e., 103 (kHz)=100 (kHz)+3 (kHz) based on the frequency data f₀ (step S115), and the SIN waveform generating circuit 54 outputs the SIN waveform of the above-described frequency. Then, the scanning circuit 50 sets the latency to 128 μsec (step S117), while continuing to output the SIN waveform.

On the other hand, when the frequency fₒᵤₜ matches with the resonance frequency fₛ in step S118 (Yes in step S118), after the operation of frequency sweep by the PLL control circuit 52, the output switch 4 is turned OFF and the output of the SIN waveform is stopped (steps S119 to S121), to end the operation. When the frequency fₒᵤₜ does not match with the resonance frequency fₛ in step S118 (No in step S118), the scanning circuit 50 decreases the frequency fout of the SIN waveform by 2 Hz (step S122), and determines whether the frequency fₒᵤₜ of the SIN waveform is lower than the lower limit of the sweeping range, i.e., 97 (kHz)=100 (kHz)−3 (kHz) or not (step S123), and if the frequency fₒᵤₜ of the SIN waveform does not reach 97 (kHz) (No in step S123), the process returns to step S116, and the SIN waveform with a pertinent frequency fₒᵤₜ=103 (kHz)−2 (Hz) is output and the above-described operation is repeated. When the frequency fₒᵤₜ of the SIN waveform is lower than 97 (kHz), the FPGA circuit 34 stops the output of the SIN waveform and outputs an error signal (step S124, S125).

When the output frequency of the handpiece 2 is 200 kHz based on the above-described identification information in step S103, similarly to the above description, the CPU 33 outputs the frequency data f₀ indicating 200 kHz to the FPGA circuit 34, and the scanning circuit 50 of the FPGA circuit 34 sets the frequency fₒᵤₜ of the SIN waveform (drive signal) to the upper limit of the sweeping range, i.e., 206 (kHz)=200 (kHz)+6 (kHz) based on the frequency data f₀ (step S126), and the SIN waveform generating circuit 54 outputs the SIN waveform of the above-described frequency (step S127). Then, the scanning circuit 50 sets the latency to 64 μsec (step S128), while maintaining the output of the SIN waveform.

Then in step S129, when the frequency fₒᵤₜ matches with the resonance frequency fₛ (Yes in step S129), after the frequency sweep operation by the PLL control circuit 52, the output switch 4 is turned OFF and the output of the SIN waveform is stopped (steps S130 to S132), to end the operation. Here in step S129, when the frequency fₒᵤₜ does not match with the resonance frequency fₛ (No in step S129), the scanning circuit 50 decreases the frequency fₒᵤₜ of the SIN waveform by 4 Hz (step S133), and determines whether the frequency fₒᵤₜ of the SIN waveform is lower than the lower limit of the sweeping range, i.e., 194 (kH)=200 (kHz)−6 (kHz) or not (step S134), and when the frequency fₒᵤₜ of the SIN waveform does not reach 194 kHz (No in step S134), the process returns to step S127 and the SIN waveform of a pertinent frequency fₒᵤₜ=206 (kHz)−4 (Hz) is output, and the above described operation is repeated. When the frequency fₒᵤₜ of the SIN waveform decreases down below 194 kHz, the FPGA circuit 34 stops the output of the SIN waveform and outputs an error signal (steps S135, S136).

Hence, in the embodiment, the setting of the sweeping range is changed for the frequency sweep according to the output frequency of the handpiece 2, and the resonance frequency at the resonance point is detected within the appropriate sweeping range, whereby dissimilar to the above-cited patent documents where the sweeping range of the resonance point is fixed for different output frequencies, misdetection of resonance point does not occur in the detection of a particularly low output frequency band, and a reliable detection of the resonance frequency at the resonance point is allowed.

Further in the embodiment, since the setting of the change width of the frequency is changed for frequency sweep according to the output frequency of the handpiece 2 to detect the resonance frequency at the resonance point in the appropriate change width, dissimilar to the apparatus where the change width of the frequency is fixed for different output frequencies, the detection accuracy is not deteriorated even in the detection of a particularly high output frequency band where a narrow frequency change width lengthens detection time, whereby the detection of resonance frequency can be realized in a short time period even in the detection of different output frequency band, and the detection accuracy can be improved.

Further, in the embodiment, since the setting of the latency for the frequency change for the frequency sweep is changed according to the output frequency of the handpiece 2, and the detection of the resonance frequency is performed at the appropriate latency, dissimilar to the apparatus where the latency is fixed for different output frequencies, the number of SIN waveforms does not increase even in the detection of a particularly high output frequency band with a long latency, whereby the detection of the resonance frequency can be realized in a short time period even in the detection of different output frequency bands, and the detection accuracy can be improved.

Next, the structure of the SIN waveform generating circuit 54 will be described.

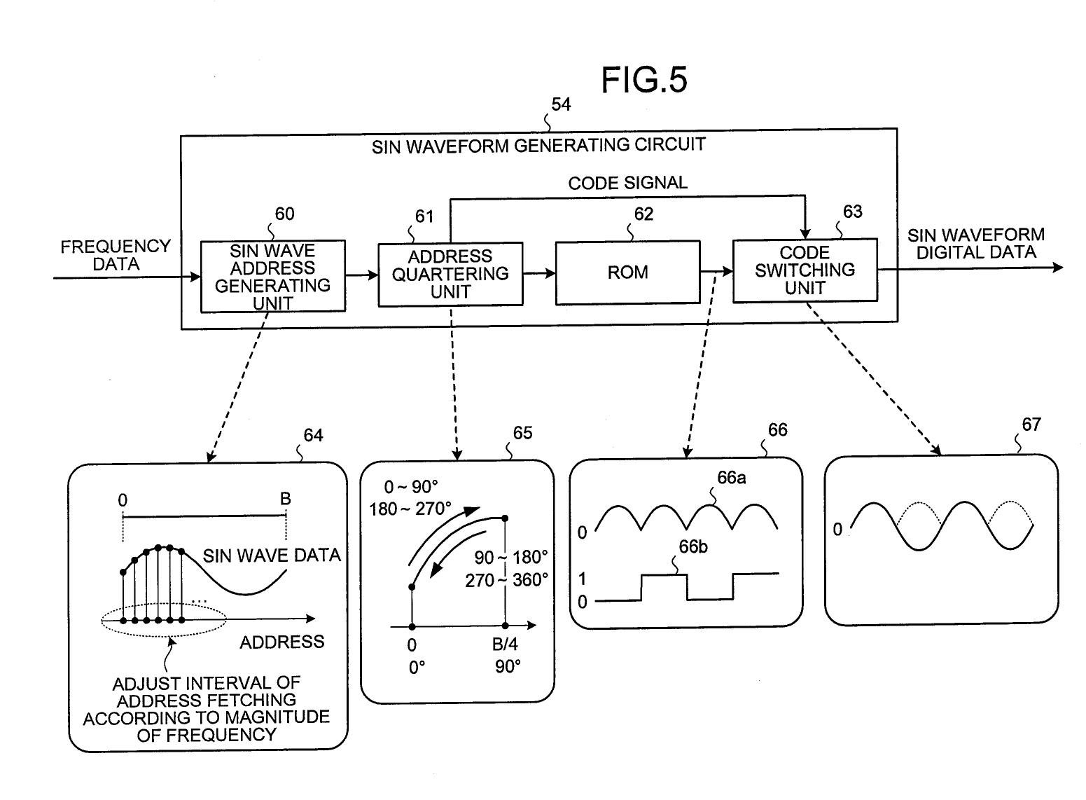

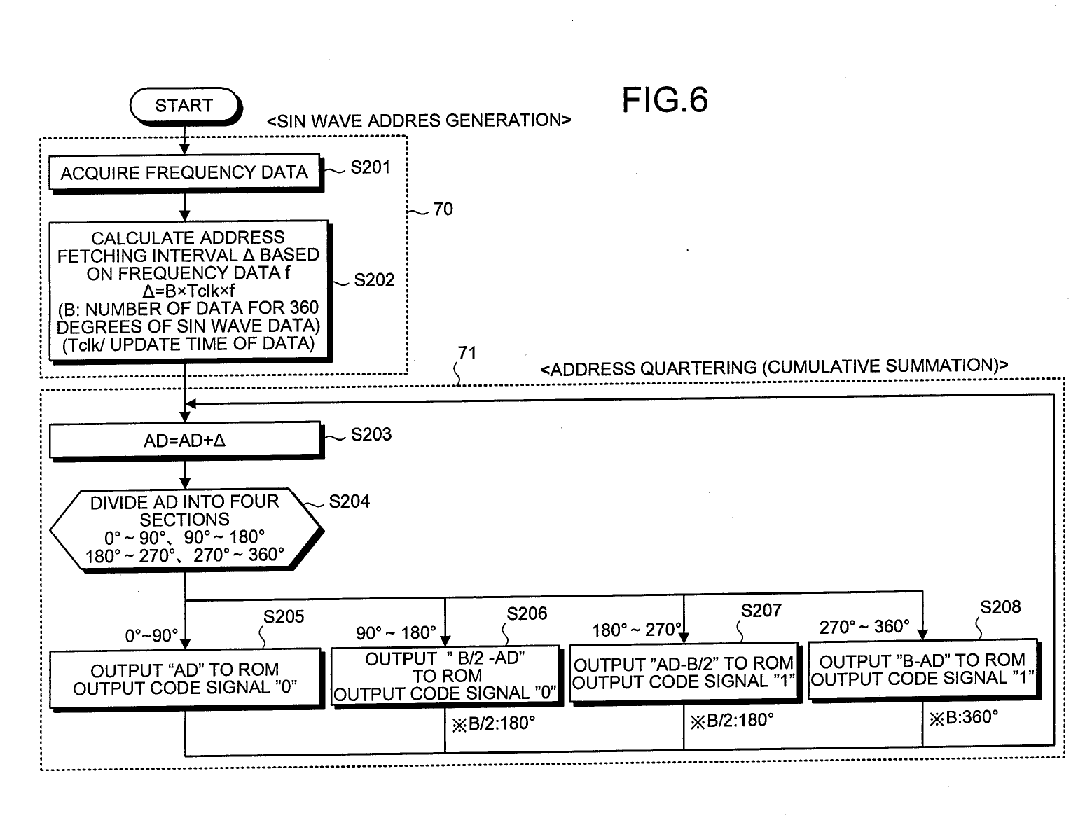

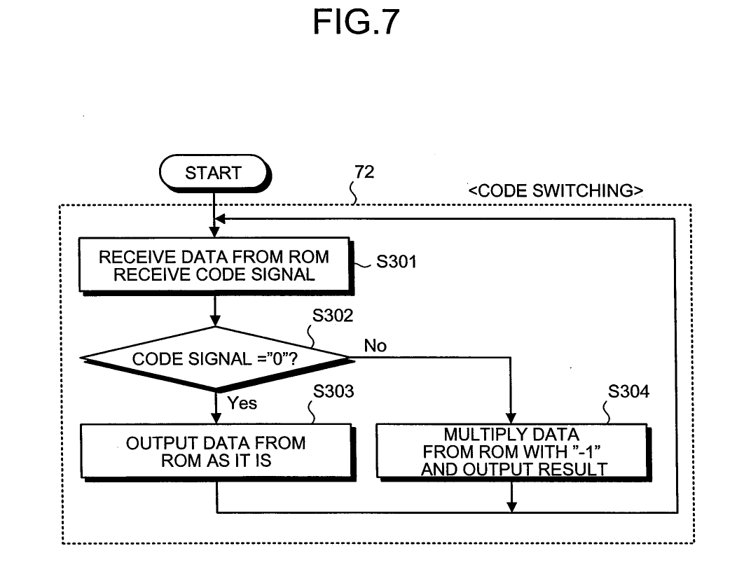

FIG. 5 is a block diagram of a structure of the SIN waveform generating circuit shown in FIG. 2. FIG. 6 is a flowchart illustrating operations of a SIN waveform generating unit and an address quartering unit shown in FIG. 5. FIG. 7 is a flowchart illustrating an operation of a code switching unit shown in FIG. 5.

Conventionally, SIN waveform digital data supplied by the SIN waveform generating circuit as an output is stored in a ROM, for example, in the SIN waveform generating circuit. Hence, when the detected resonance frequencies exist in a wide range of frequency band, SIN waveform digital data must be prepared in accordance with all resonance frequencies to be detected, and such SIN waveform digital data needs to be stored in the ROM. In particular, when a highly accurate formulation of the SIN waveform digital data is desirable, the ROM needs to have a large amount of capacity to accommodate a large amount of data, whereby data amount is increased resulting in the necessity of a high capacity memory to store such data.

The present invention at least solves the problem as described above.

As shown in FIG. 5, the SIN waveform generating circuit 54 includes a SIN wave address generating unit 60, an address quartering unit 61, a ROM 62, and a code switching unit 63. The SIN wave address generating unit 60 serves to adjust the interval of address fetching according to the magnitude of detected frequency as shown in a block 64 of FIG. 5. The SIN wave address generating unit 60 adjusts the interval of address fetching to a longer interval on generating a low-frequency SIN wave data, whereas adjusts the interval of address fetching to a shorter interval on generating a high-frequency SIN wave data, for example, for the address generation of the SIN wave data.

The ROM 62 stores SIN wave data corresponding to a quarter cycle, 0 to B/4, for the SIN wave data of one cycle of 0 to B, for example, as shown in a block 65 of FIG. 5, and the address quartering unit 61 accumulates the SIN wave data corresponding to the quarter time period stored in the ROM 62, reads out the waveform data shown in a block 66 of FIG. 5, for generation.

The code switching unit 63 performs a code switching on the waveform data read out from the ROM 62 to generate and output the SIN waveform digital data as shown in a block 67 of FIG. 5.

Next, a generation operation of the SIN waveform digital data in the SIN waveform generating circuit 54 will be described with reference to the flowcharts of FIGS. 6 and 7. In FIG. 6, the SIN wave address generating unit 60 performs the SIN wave address generation shown in a block 70, and the address quartering unit 61 performs an address quartering (cumulative summation method) of a block 71. First, the SIN wave address generating unit 60 acquires a frequency data f (drive signal fₛ or fᵣ) from the frequency changing circuit 57 or the PLL control circuit 52 (step S201), and calculates an address fetching interval Δ based on the frequency data f (step S202). Here, the address fetching interval Δ can be represented as Δ=B×T_{clk}×f, where B is a number of data corresponding to 360 degrees of SIN wave data, and T_{clk} is an update timing of the waveform data read out from the ROM 62. Hence, when the values B and T_{clk} are constant, for example, when the acquired frequency data f has a high frequency, the address fetching interval Δ is long, whereas when the frequency data f has a low frequency, the address fetching interval Δ is short.

The address quartering unit 61 sets the address interval by setting an address AD of the waveform data retrieved from the ROM 62 as AD=AD+Δ, based on the address fetching interval Δ supplied from the SIN wave address generating unit 60 (step S203). Next, the interval of address AD reading is divided into four sections, i.e., 0 to 90 degrees, 90 to 180 degrees, 180 to 270 degrees, and 270 to 360 degrees (step S204).

Here, as shown in the block 65 of FIG. 5, in the section of 0 to 90 degrees, the address AD is sequentially output to the ROM at the address fetching interval Δ starting from 0 degree as a base point “0” up to 90 degrees, and a code signal “0” instructing prohibition of waveform data inversion is supplied to the code switching unit 63 (step S205). After the completion of address fetching from the section of 0 to 90 degrees, the address quartering unit 61 proceeds to the address output from the section of 90 to 180 degrees.

In the address output from the section of 90 to 180 degrees, the address AD is sequentially output to the ROM at the address fetching interval Δ starting from 180 degrees as a base point “0” up to 90 degrees, and the code signal “0” instructing prohibition of waveform data inversion is supplied to the code switching unit 63 (step S206). Here, until the process reaches 180 degrees in “B/2−AD,” the address is read out in a reverse order from the address reading from the section 0 to 90 degrees and output to the ROM. After the completion of the address fetching from the section of 90 to 180 degrees, the address quartering unit 61 performs the address output from the section of 180 to 270 degrees.

In the address output from the section of 180 to 270 degrees, the address AD is sequentially output to the ROM at the address fetching interval Δ starting from 180 degrees as a base point “0” up to 270 degrees, and a code signal “1” instructing waveform data inversion is supplied to the code switching unit 63 (step S207). Here, until the process reaches 270 degrees in “AD−B/2,” the address is read out similarly to the address reading from the section of 0 to 90 degrees and output to the ROM. After the completion of the address fetching from the section of 180 to 270 degrees, the address quartering unit 61 performs the address output from the section of 270 to 360 degrees.

In the address output from the section of 270 to 360 degrees, the address AD is sequentially output to the ROM at the address fetching interval Δ starting from 360 degrees as a base point “0” up to 270 degrees, and the code signal “1” instructing waveform data inversion is supplied to the code switching unit 63 (step S208). Here, until the process reaches 360 degrees in “B−AD,” the address is read out in a reverse order from the address reading from the section of 0 to 90 degrees and output to the ROM. After the completion of the address fetching from the section of 270 to 360 degrees, the address quartering unit 61 returns to step S203 and repeats the operation described above.

Through the address reading operation of the address quartering unit 61, a waveform data 66a of one cycle formed with two upward curves starting from “0” as a base point as shown in a block 66, and a clock signal 66b with a waveform of “0” and “1” indicating a time length of one cycle are output from the ROM 62 to the code switching unit 63. Here, in the block 66, the waveform data 66a and the clock signal 66b of two cycles are shown.

In FIG. 7, the code switching unit 63 receives the waveform data 66a and the clock signal 66b from the ROM 62 and receives the code signal from the address quartering unit 61 (step S301), to determine whether the code signal is “0” or not (step S302).

Here, if the code signal is “0” (Yes in step S302), the code switching unit 63 outputs the waveform data 66a from the ROM as it is to the D/A converter 35 as SIN waveform digital data (step S303). On the other hand, if the code signal is not “0” (No in step S302), the code switching unit 63 multiplies the waveform data 66a from the ROM by “−1” to invert the data and output the result to the D/A converter 35 (step S304). Thus, the SIN waveform digital data (shown by a solid line in the drawing) is output from the code switching unit 63 as shown in the block 67 of FIG. 5.

Thus, the SIN waveform generating circuit 54 performs generation and output of the SIN waveform corresponding to the drive signal fₛ or fᵣ supplied from the frequency changing circuit 57 or the PLL control circuit 52. The digital data of the SIN waveform supplied as an output from the SIN waveform generating circuit 54 is converted into analog data by the D/A converter 35, which result is amplified by the amplifier 37, and the result is output to the ultrasonic transducer 21 in the handpiece 2.

Thus in the embodiment, the SIN waveform for each frequency band is generated based on the waveform data of a quarter amount of the SIN waveform of one cycle, and therefore the data amount the ROM is required to store is only a quarter the data of SIN waveform of one cycle, whereby the memory capacity of the ROM can be decreased. Thus, a low-cost low-capacity ROM is applicable as to realize reduced manufacturing cost of the ultrasonic surgical apparatus.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An electronic treatment apparatus comprising:
a handpiece having an ultrasonic transducer configured and operative to generate ultrasonic vibrations, and an identification storing unit in which identification information specific to the ultrasonic transducer is stored, the identification information including output frequency data and resonance frequency data of the ultrasonic transducer;
an identification detecting unit that detects the identification information from identification storing unit;
a sweeping rage setting unit configured and operative to set a sweeping rage of frequencies a drive signal for driving the ultrasonic transducer based on the detected identification information; and
a resonance frequency detecting unit configured and operative to perform a frequency sweep on the drive signal in the sweeping rage to detect a resonance frequency of the ultrasonic transducer.

2. The ultrasonic treatment apparatus according to claim 21, further comprising:
a frequency change width setting unit configured and operative to set a change width of the frequency to be swept in the frequency sweep based on the identification information.

3. The ultrasonic treatment apparatus according to claim 21, further comprising:
a tie interval setting unit configured and operative to set a tie interval for changing one frequency to another frequency of the drive sign in the frequency sweep based on the identification information.

4. The ultrasonic treatment apparatus according to claim 21, further comprising:
a frequency change width setting unit configured and operative to set a change width of the frequency to be swept in the frequency sweep based on the identification information;
a tie interval setting unit configured and operative to set a tie interval for changing one frequency to another frequency of the drive signal in the frequency sweep based on the identification information; and
a frequency condition setting unit configured and operative to
set the sweeping rage and the change width to be smaller and the tie interval to be longer if the output frequency data of the identification information indicates a frequency band lower than a predetermined frequency, and
set the sweeping range and the change width to be larger and the tie interval to be shorter if the output frequency data of the identification information indicates a frequency band higher than the predetermined frequency.
